(19) United States
(12) Patent Application Publication
(10) Pub. No.: US 2012/0178675 A1
(43) Pub. Date: Jul. 12, 2012

(54) Compositions And Methods For Modulating The Pharmacokinetics and Pharmacodynamics of Insulin

(75) Inventors: Roderike Pohl, Sherman, CT (US); Solomon Steiner, Mount Kisco, NY (US); Robert Hauser, Columbia, MD (US); Richard Seibert, Carmel, NY (US); Ming Li, Yorktown Heights, NY (US)

(73) Assignee: Biodel Inc.

(21) Appl. No.: 13/176,435

(22) Filed: Jul. 5, 2011

Related U.S. Application Data
(60) Provisional application No. 61/361,980, filed on Jul. 7, 2010.
(60) Provisional application No. 61/381,492, filed on Sep. 10, 2010.
(60) Provisional application No. 61/433,080, filed on Jan. 14, 2011.
(60) Provisional application No. 61/484,553, filed on May 10, 2011.

(51) Int. Cl. A61K 38/28; A61P 25/00; A61P 3/10

(52) U.S. Cl. 514/6.4

(57) ABSTRACT

Compositions and methods for modulating the pharmacokinetics and pharmacodynamics of rapid acting injectable insulin formulations are described herein. In the preferred embodiment, the formulations are administered via subcutaneous injection. The formulations contain insulin in combination with a zinc chelator such as ethylenediaminetetraacetic acid (“EDTA”) and a dissolution/stabilization agent, and optionally additional excipients. Calcium disodium EDTA is less likely to remove calcium from the body, and typically has less pain on injection in the subcutaneous tissue. Modulating the type and quantity of EDTA can change the insulin absorption profile. Increasing the quantity of citrate can further enhance absorption and chemically stabilize the formulation. In the preferred embodiment, the formulation contains human insulin, calcium disodium EDTA and a dissolution/stabilization agent such as citric acid or sodium citrate. These formulations are rapidly absorbed into the blood stream when administered by subcutaneous injection.

### CROSS REFERENCE TO RELATED APPLICATIONS

This application claims benefit of and priority to U.S. Ser. No. 61/361,980 filed Jul. 7, 2010; U.S. Ser. No. 61/381,492 filed Sep. 10, 2010; U.S. Ser. No. 61/433,080 filed Jan. 14, 2011; and U.S. Ser. No. 61/494,553 filed May 10, 2011, all of which are incorporated herein by reference in their entirety.

### FIELD OF THE INVENTION

The invention is in the general field of injectable rapid acting drug delivery insulin formulations and methods of their use and reduction of pain on injection.

### BACKGROUND OF THE INVENTION

Diabetes Overview

Glucose is a simple sugar used by all the cells of the body to produce energy and support life. Humans need a minimum level of glucose in their blood at all times to stay alive. The primary manner in which the body produces blood glucose is through the digestion of food. When a person is not getting this glucose from food digestion, glucose is produced from stores in the tissue and released by the liver. The body's glucose levels are regulated by insulin. Insulin is a peptide hormone that is naturally secreted by the pancreas. Insulin helps glucose enter the body's cells to provide a vital source of energy.

When a healthy individual begins a meal, the pancreas releases a natural spike of insulin called the first-phase insulin release. In addition to providing sufficient insulin to process the glucose coming into the blood from digestion of the meal, the first-phase insulin release acts as a signal to the liver to stop making glucose while digestion of the meal is taking place. Because the liver is not producing glucose and there is sufficient additional insulin to process the glucose from digestion, the blood glucose levels of healthy individuals remain relatively constant and their blood glucose levels do not become too high.

Diabetes is a disease characterized by abnormally high levels of blood glucose and inadequate levels of insulin. There are two major types of diabetes—Type 1 and Type 2. In Type 1 diabetes, the body produces no insulin. In the early stages of Type 2 diabetes, although the pancreas does produce insulin, either the body does not produce the insulin at the right time or the body's cells ignore the insulin, a condition known as insulin resistance.

Even before any other symptoms are present, one of the first effects of Type 2 diabetes is the loss of the meal-induced first-phase insulin release. In the absence of the first-phase insulin release, the liver will not receive its signal to stop making glucose. As a result, the liver will continue to produce glucose at a time when the body begins to produce new glucose through the digestion of the meal. As a result, the blood glucose level of patients with diabetes goes too high after eating, a condition known as hyperglycemia. Hyperglycemia causes glucose to attach unnaturally to certain proteins in the blood, interfering with the proteins' ability to perform their normal function of maintaining the integrity of the small blood vessels. With hyperglycemia occurring after each meal, the tiny blood vessels eventually break down and leak. The long-term adverse effects of hyperglycemia include blindness, loss of kidney function, nerve damage and loss of sensation and poor circulation in the periphery, potentially requiring amputation of the extremities.

Between two and three hours after a meal, an untreated diabetic's blood glucose becomes so elevated that the pancreas receives a signal to secrete an inordinately large amount of insulin. In a patient with early Type 2 diabetes, the pancreas can still respond and secretes this large amount of insulin. However, this occurs at the time when digestion is almost over and blood glucose levels should begin to fall. This inordinately large amount of insulin has two detrimental effects. First, it puts an undue extreme demand on an already compromised pancreas, which may lead to its more rapid deterioration and eventually render the pancreas unable to produce insulin. Second, too much insulin after digestion leads to weight gain, which may further exacerbate the disease condition.

Current Treatments for Diabetes and Their Limitations

Because patients with Type 1 diabetes produce no insulin, the primary treatment for Type 1 diabetes is daily intensive insulin therapy. The treatment of Type 2 diabetes typically starts with management of diet and exercise. Although helpful in the short-run, treatment through diet and exercise alone is not an effective long-term solution for the vast majority of patients with Type 2 diabetes. When diet and exercise are no longer sufficient, treatment commences with various non-insulin oral medications. These oral medications act by increasing the amount of insulin produced by the pancreas, by increasing the sensitivity of insulin-sensitive cells, by reducing the glucose output of the liver or by some combination of these mechanisms. These treatments are limited in their ability to manage the disease effectively and generally have significant side effects, such as weight gain and hypertension. Because of the limitations of non-insulin treatments, many patients with Type 2 diabetes deteriorate over time and eventually require insulin therapy to support their metabolism.

Insulin therapy has been used for more than 80 years to treat diabetes. This therapy usually involves administering several injections of insulin each day. These injections consist of administering a long-acting basal injection one or two times per day and an injection of a fast acting insulin at meal-time. Although this treatment regimen is accepted as effective, it has limitations. First, patients generally dislike injecting themselves with insulin due to the inconvenience and pain of needles. As a result, patients tend not to comply adequately with the prescribed treatment regimens and are often improperly medicated.

More importantly, even when properly administered, insulin injections do not replicate the natural time-action profile of insulin. In particular, the natural spike of the first-phase insulin release in a person without diabetes results in blood insulin levels rising within several minutes of the entry into the blood of glucose from a meal. By contrast, injected insulin enters the blood slowly, with peak insulin levels occurring within 80 to 100 minutes following the injection of regular human insulin.

A potential solution is the injection of insulin directly into the vein of diabetic patients immediately before eating a meal. In studies of intravenous injections of insulin, patients exhibited better control of their blood glucose for 3 to 6 hours following the meal. However, for a variety of medical reasons, intravenous injection of insulin before each meal is not a practical therapy.

One of the key improvements in insulin treatments was the introduction in the 1990s of rapid-acting insulin analogs, such as Humalog®, Novolog® and Apidra®. However, even with the rapid-acting insulin analogs, peak insulin levels typically occur within 50 to 70 minutes following the injection. Because the rapid-acting insulin analogs do not adequately mimic the first-phase insulin release, diabetics using insulin therapy continue to have inadequate levels of insulin present at the initiation of a meal and too much insulin present between meals. This lag in insulin delivery can result in hyperglycemia early after meal onset. Furthermore, the excessive insulin between meals may result in an abnormally low level of blood glucose known as hypoglycemia. Hypoglycemia can result in loss of mental acuity, confusion, increased heart rate, hunger, sweating and faintness. At very low glucose levels, hypoglycemia can result in loss of consciousness, coma and even death. According to the American Diabetes Association, or ADA, insulin-using diabetic patients have on average 1.2 serious hypoglycemic events per year, many of which events require hospital emergency room visits by the patients.

The rapidity of insulin action is dependent on how quickly it is absorbed. When regular human insulin is injected subcutaneously at relatively high concentrations (100 IU/ml), the formulation is primarily composed of hexamers (approximately 36 kDa) which are not readily absorbed due to their size and charge. Located within the hexamer are two zinc atoms that stabilize the molecule. Post injection, a concentration driven dynamic equilibrium occurs in the subcutaneous tissue causing the hexamers to dissociate into dimers (about 12 kDa), then monomers(about 6 kDa). Historically, these regular human insulin formulations require approximately 120 min. to reach maximum plasma concentration levels.

Insulin formulations with a rapid onset of action, such as VIAject®, are described in U.S. Pat. No. 7,279,457, and U.S. Published Applications 2007/0235365, 2008/0085298, 2008/90753, and 2008/0096800, and Steiner, et al., Diabetologia, 51:1602-1606 (2008). The rapid acting insulin formulations were designed to create insulin formulations that provide an even more rapid pharmacokinetic profile than insulin analogs, thereby avoiding the patient becoming hyperglycemic in the first hour after injection and hypoglycemic two to four hours later. The rapid onset of VIAject® results from the inclusion of two key excipients, a zinc chelator such as disodium EDTA (EDTA) or calcium disodium EDTA which rapidly dissociates insulin hexamers into monomers and dimers and a dissolution/stabilization agent such as citric acid which stabilizes the monomers and dimers prior to being absorbed into the blood (Pohl, et al., presented at Controlled Release Society 36^{th} annual meeting (2009). Unfortunately, early clinical trials with this product showed some injection site discomfort.

It is an object of the present invention to provide specific insulin formulations for treating a diabetic which modulate the pharmacokinetics and pharmacodynamics of injectable insulin compositions.

It is a further object of this invention to provide compositions of rapid acting injectable insulin compositions with reduced injection site discomfort and enhanced shelf life (stability).

### SUMMARY OF THE INVENTION

Compositions and methods for modulating the pharmacokinetics and pharmacodynamics of rapid acting injectable insulin formulations and reducing site reactions are described herein. In the preferred embodiment, the formulations are administered via subcutaneous injection. The formulations contain insulin in combination with a zinc chelator such as ethylenediamine tetraacetic acid (“EDTA”) and a dissolution/stabilization agent such as citric acid and/or sodium citrate, and optionally additional excipients. EDTA comes in two injectable forms, disodium EDTA and calcium disodium EDTA. Calcium disodium EDTA is less likely to remove calcium from the body, and typically has less pain on injection in the subcutaneous tissue. The preferred range of calcium disodium EDTA is 6 mg-0.2 mg/mL. The most preferred range is from 4-1 mg/mL. In the preferred embodiment, the formulation contains recombinant human insulin, calcium disodium EDTA and a dissolution/stabilization agent such as citric acid and/or sodium citrate. Stability is enhanced by optimizing m-cresol and citrate ion concentration.

Compositions and methods for optimizing the rate of insulin absorption and time to decrease the blood glucose levels in a diabetic individual have been developed wherein the chelator form and concentration is varied to produce different absorption profiles and reduction of injection site pain.

### BRIEF DESCRIPTION OF THE DRAWINGS

FIG. 1 is a three dimensional schematic of insulin showing exposed surface charges and overlaid with molecules (“dissolution and chelating agents”) of appropriate size to mask the charge.

FIG. 2 is a graph of mean insulin concentration over time for eight miniature diabetic swine (dose 0.25 U/kg) for the first 100 min. post injection. EDTA concentrations in formulations are 1.8 mg/mL (VJ7, solid diamond), 1.0 mg/mL (VV1, open square), 0.25 mg/mL (VV3, solid triangle) and 0.1 mg/mL (VV4, open circle with dotted line), +/− SEM.

FIG. 3 is a graph of mean insulin concentration over time for eight miniature diabetic swine (dose 0.25 U/kg).for the first 250 min. post injection. BIOD 105 (open diamond), BIOD 107 (solid square) vs. VJ7 (triangle, dotted line).

### DETAILED DESCRIPTION OF THE INVENTION

The insulin formulations are administered immediately prior to a meal or at the end of a meal. The formulations are designed to be absorbed into the blood faster than the currently marketed rapid-acting insulin analogs. One of the key features of the formulation of insulin is that it disassociates, or separates, the hexameric form of insulin to the monomeric form of insulin and prevents re-association to the hexameric form post injection, thereby promoting rapid absorption into the bloodstream post injection.

It has been discovered that a systematic relationship exists between the concentration of zinc chelator, such as disodium EDTA, and the speed of glucose absorption from the blood. Variation in EDTA concentration alters the pharmacokinetics and pharmacodynamics of rapid acting insulin formulations.

A possible reason for the injection site discomfort of the EDTA-citric acid-insulin formulation is chelation of extracellular calcium by disodium EDTA. Calcium is in the extracellular fluid at a concentration of approximately 1 mM, and is essential for excitation-contraction coupling, muscle function, neurotransmitter release, and cellular metabolism. Loss of local calcium can cause muscle tetany, which is a disorder marked by intermittent tonic muscular contractions, accompanied by fibrillary tremors, paresthesias and muscular pain. To avoid this interaction, calcium should not be removed from the extracellular fluid.

The substitution of the calcium chelated form of EDTA (calcium disodium EDTA) reduces injection site pain as compared to the same amount of disodium EDTA. However, calcium disodium EDTA slightly delays the rate of absorption in vivo. It is possible to obtain an equivalent rate of absorption to that seen with disodium EDTA by using more calcium disodium EDTA, for example, 120%, as compared to disodium EDTA. Therefore, changes in the concentration and form of EDTA can be used to fine-tune rapid acting insulin formulations to a desired pharmacokinetic and pharmacodynamic profile, and improve site pain post injection.

Injection site tolerability and stability of the calcium disodium EDTA insulin formulations can also be enhanced by the method of preparation. In the preferred embodiment, the insulin hexamer is dissociated by addition of calcium disodium EDTA to the insulin. In another preferred embodiment, calcium chloride and disodium EDTA is added. The added calcium complexes with the EDTA, reducing the interaction of the EDTA with the interstitial calcium. In yet another embodiment, additional citrate ions are used to enhance the rapid uptake of the formulation. In addition, m-cresol concentration was reduced, which enhanced the shelf life (stability).

### I. Definitions

As used herein, “insulin” refers to human or non-human, recombinant, purified or synthetic insulin or insulin analogues, unless otherwise specified.

As used herein, “Human insulin” is the human peptide hormone secreted by the pancreas, whether isolated from a natural source or made by genetically altered microorganisms. As used herein, “non-human insulin” is the same as human insulin but from an animal source such as pig or cow.

As used herein, an insulin analogue is an altered insulin, different from the insulin secreted by the pancreas, but still available to the body for performing the same action as natural insulin. Through genetic engineering of the underlying DNA, the amino acid sequence of insulin can be changed to alter its ADME (absorption, distribution, metabolism, and excretion) characteristics. Examples include insulin lispro, insulin glargine, insulin aspart, insulin glulisine, and insulin detemir. The insulin can also be modified chemically, for example, by acetylation. As used herein, human insulin analogues are altered human insulin which is able to perform the same action as human insulin.

As used herein, a “chelator” or “chelating agent”, refers to a chemical compound that has the ability to form one or more bonds to zinc ions. The bonds are typically ionic or coordination bonds. The chelator can be an inorganic or an organic compound. A chelate complex is a complex in which the metal ion is bound to two or more atoms of the chelating agent.

As used herein, a “solubilizing agent”, is a compound that increases the solubility of materials in a solvent, for example, insulin in an aqueous solution. Examples of solubilizing agents include surfactants such as TWEEN®; solvents such as ethanol; micelle forming compounds, such as oxyethylene monostearate; and pH-modifying agents.

As used herein, a “dissolution/stabilization agent” or “dissolution/stabilizing agent” is an acid or a salt thereof that, when added to insulin and EDTA, enhances the transport and absorption of insulin relative to HCl and EDTA at the same pH, as measured using the epithelial cell transwell plate assay described in the examples below. HCl is not a dissolution/stabilization agent but may be a solubilizing agent. Citric acid is a dissolution/stabilization agent when measured in this assay.

As used herein, an “excipient” is an inactive substance other than a chelator or dissolution/stabilization agent, used as a carrier for the insulin or used to aid the process by which a product is manufactured. In such cases, the active substance is dissolved or mixed with an excipient.

As used herein, a “physiological pH” is between 6.8 and 7.6, preferably between 7 and 7.5, most preferably about 7.4.

As used herein, “Cmax” is the maximum or peak concentration of a drug observed after its administration.

As used herein, “Tmax” is the time at which maximum concentration (Cmax) occurs.

As used herein, ½ Tmax is the time at which half maximal concentration (½ Cmax) of insulin occurs in the blood.

### II. Formulations

Formulations include insulin, a chelator and a dissolution/.stabilizing agent(s) and, optionally, one or more other excipients. In the preferred embodiment, the formulations are suitable for subcutaneous administration and are rapidly absorbed into the fatty subcutaneous tissue. The choice of dissolution/stabilization agent and chelator, the concentration of both the dissolution/stabilization agent and the chelator, and the pH that the formulation is adjusted to, all have a profound effect on the efficacy of the system. While many combinations have efficacy, the preferred embodiment is chosen for reasons including safety, comfort, stability, regulatory profile, and performance.

In the preferred embodiment, at least one of the formulation ingredients is selected to mask charges on the insulin. This may facilitate the transmembrane transport of the insulin and thereby increase both the onset of action and bioavailability for the insulin. The ingredients are also selected to form compositions that dissolve rapidly in aqueous medium. Preferably the insulin is absorbed and transported to the plasma quickly, resulting in a rapid onset of action, preferably beginning within about 5 minutes following administration and peaking at about 15-30 minutes following administration.

The chelator, such as EDTA, chelates the zinc within the insulin, thereby removing the zinc from the insulin molecule. This causes the hexameric insulin to dissociate into its dimeric and monomeric forms and retards reassembly into the hexamer state post injection. Since these two forms exist in a concentration-driven equilibrium, as the monomers are absorbed, more monomers are created. Thus, as insulin monomers are absorbed through the subcutaneous tissue, additional dimers dissemble to form more monomers. The monomeric form has a molecular weight that is less than one-sixth the molecular weight of the hexameric form, thereby markedly increasing both the speed and quantity of insulin absorption. To the extent that the chelator (such as EDTA) and/or dissolution/stabilization agent (such as citric acid) hydrogen bond with the insulin, it is believed that they mask the charge on the insulin, facilitating its transmembrane transport and thereby increasing both the onset of action and bioavailability of the insulin.

Injection site tolerability and stability of the calcium disodium EDTA insulin formulations can also be enhanced by the method of preparation. In the preferred embodiment, the insulin hexamer is dissociated by addition of calcium or calcium and sodium disodium EDTA to the insulin. However, the calcium disodium EDTA tends to retard the rapid uptake of the formulation. Alternatives to the direct addition of CaEDTA have shown that the rapid absorption can be achieved by substitution of disodium EDTA and CaCl₂, and increasing the amount of sodium citrate. The calcium chloride is added to the formulation to “neutralize” disodium EDTA, reducing its interaction with interstitial calcium which creates the site reaction. Sodium citrate and/or citric acid is added in higher concentrations to enhance the absorption of insulin.

M-cresol is added for its anti-microbial properties and enhancement of shelf life.

Insulin

Insulin or insulin analogs may be used in this formulation. Preferably, the insulin is recombinant human insulin. Recombinant human insulin is available from a number of sources. The dosages of the insulin depend on its bioavailability and the patient to be treated. Insulin is generally included in a dosage range of 1.5-100 IU, preferably 3-50 IU per human dose. Typically, insulin is provided in 100 IU vials. In the most preferred embodiment the injectable formulation is a volume of 1 ml containing 100 U of insulin.

Dissolution/Stabilization Agents

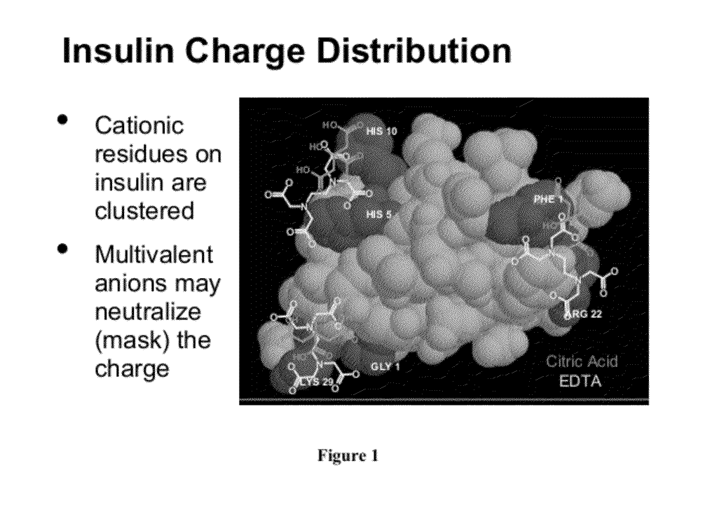

Certain polyacids appear to mask charges on the insulin, enhancing uptake and transport, as shown in FIG. 1. Those acids which are effective as dissolution/stabilization agents include acetic acid, ascorbic acid, citric acid, glutamic acid, aspartic acid, succinic acid, fumaric acid, maleic acid, adipic acid, and salts thereof, relative to hydrochloric acid. For example, if the active agent is insulin, a preferred dissolution/stabilization agent is citric acid and/or sodium citrate. Hydrochloric acid may be used for pH adjustment, in combination with any of the formulations, but is not a dissolution/stabilization agent.

Salts of the acids include sodium acetate, ascorbate, citrate, glutamate, aspartate, succinate, fumarate, maleate, and adipate. Salts of organic acids can be prepared using a variety of bases including, but not limited to, metal hydroxides, metal oxides, metal carbonates and bicarbonates, metal amines, as well as ammonium bases, such as ammonium chloride, ammonium carbonate, etc. Suitable metals include monovalent and polyvalent metal ions. Exemplary metals ions include the Group I metals, such as lithium, sodium, and potassium; Group II metals, such as barium, magnesium, calcium, and strontium; and metalloids such as aluminum. Polyvalent metal ions may be desirable for organic acids containing more than carboxylic acid group since these ions can simultaneously complex to more than one carboxylic acid group.

The range of dissolution/stabilization agent corresponds to an effective amount of citric acid in combination with insulin and disodium EDTA. For example, a range of 9.37×10⁻⁴ M to 9.37×10⁻²M citric acid corresponds with a weight/volume of about 0.18 mg/ml to about 18 mg/ml if the citric acid is anhydrous citric acid with a molar mass of approximately 192 gram/mole. In some embodiments the amount of anhydrous citric acid ranges from about 50% of 1.8 mg/ml (0.9 mg/ml) to about 500% of 1.8 mg/ml (9 mg/ml), more preferably from about 75% of 1.8 mg/ml (1.35 mg/ml) to about 300% of 1.8 mg/ml (5.4 mg/ml). In a preferred embodiment, the amount of anhydrous citric acid can be about 1.8 mg/ml, or about 2.7 mg/ml, or about 3.6 mg/ml, or about 5.4 mg/ml. In the most preferred embodiment, the amount of citric acid is 2.7 mg/ml of the injectable formulation. The weight/volume may be adjusted, if for example, citric acid monohydrate or trisodium citrate or another citric acid is used instead of anhydrous citric acid.

The preferred dissolution/stabilization agent when the insulin formulation has a pH in the physiological pH range is sodium citrate.

In a particularly preferred embodiment, the formulation contains a mixture of calcium disodium EDTA and citric acid. The formulation that was previously developed containing Na EDTA and citric acid. Based on values for a Na EDTA and citric acid containing formulation, in general the ratio of citric acid to calcium disodium EDTA is in the range of 300:100, for example, 100:120, 100:100, 200:100, 150:100, and 300:200.

Chelators

In the preferred embodiment, a zinc chelator is mixed with the insulin. The chelator may be ionic or non-ionic. Chelators include ethylenediaminetetraacetic acid (EDTA), EGTA, alginic acid, alpha lipoic acid, dimercaptosuccinic acid (DMSA), CDTA (1,2-diaminocyclohexanetetraacetic acid), and trisodium citrate (TSC). Hydrochloric acid is used in conjunction with TSC to adjust the pH, and in the process gives rise to the formation of citric acid, which is a dissolution/stabilization agent.

The chelator captures the zinc from the insulin, thereby favoring the monomeric or dimeric form of the insulin over the hexameric form and facilitating absorption of the insulin by the tissues surrounding the site of administration (e.g. mucosa, or fatty tissue). In addition, the chelator hydrogen may bond to the insulin, thereby aiding the charge masking of the insulin monomers and facilitating transmembrane transport of the insulin monomers.

In the preferred embodiment, the chelator is EDTA. EDTA comes in two injectable forms, disodium EDTA and calcium disodium EDTA. Disodium EDTA is provided intravenously for hypercalcemia, while calcium disodium EDTA is used as a rescue drug to treat heavy metal poisoning. Calcium disodium EDTA is less likely to remove calcium from the body, and typically has less pain on injection in the subcutaneous tissue. In one preferred embodiment, the formulation contains insulin, calcium disodium EDTA and a dissolution/stabilization agent such as citric acid or sodium citrate. In another preferred embodiment, the formulation contains insulin, disodium EDTA, calcium chloride, and a dissolution/stabilization agent such as citric acid or sodium citrate.

A range of 2.42×10⁻⁴ M to 9.68×10⁻² M EDTA corresponds to a weight/volume of about 0.07 mg/ml to about 28 mg/ml if the EDTA is Ethylenediaminetetraacetic acid with a molar mass of approximately 292 grams/mole. Reduction of the concentration of EDTA can slow the rate of insulin absorption and delay the glucose response to the insulin injection. Further increases in this concentration provide negligible gains in absorption rate.

In preferred embodiments the amount of EDTA ranges from about 5% of 1.8 mg/ml (0.09 mg/ml) to about 500% of 1.8 mg/ml (9 mg/ml), more preferably about 15% of 1.8 mg/ml (0.27 mg/ml) to about 200% of 1.8 mg/ml (3.6 mg/ml). For example, the amount of EDTA can be 0.1 mg/ml, 0.25 mg/ml, 1.0 mg/ml, 1.8 mg/ml, 2.0 mg/ml, or 2.4 mg/ml of EDTA.

Reduction of the concentration of EDTA can slow the rate of insulin absorption and delay the glucose response to the insulin injection. In a preferred embodiment the chelator is disodium EDTA, in an amount equal to or less than 2.0 mg/ml. Further increases in this concentration provide negligible gains in absorption rate. In a preferred embodiment, the chelator is calcium disodium EDTA, which can also be used to modulate the insulin absorption rate and reduce injection site pain. The preferred range of this form of EDTA is higher, since more calcium disodium EDTA is required to maximize the fast absorption of insulin. The range is 0.2-6.0 mg/ml. The preferred range is from 1-4 mg/mL.

In some embodiments, the EDTA is a combination of disodium EDTA and calcium disodium EDTA. For example, in one embodiment, the EDTA is about 0.27-0.3 mg/ml of disodium EDTA in combination with about 1.8-2.0 mg/ml of calcium disodium EDTA. In the most preferred embodiment, the EDTA is between about 1.8-2.0 mg/ml of calcium disodium EDTA or disodium EDTA and CaCl₂.

Excipients

Pharmaceutical compositions may be formulated in a conventional manner using one or more physiologically acceptable carriers comprising excipients and auxiliaries which facilitate processing of the active compounds into preparations which can be used pharmaceutically. Formulation of drugs is discussed in, for example, Hoover, John E., Remington's Pharmaceutical Sciences, Mack Publishing Co., Easton, Pa. (1975), and Liberman, H. A. and Lachman, L., Eds., Pharmaceutical Dosage Forms, Marcel Decker, New York, N.Y. (1980).

In the preferred embodiment, one or more solubilizing agents are included with the insulin to promote rapid dissolution in aqueous media. Suitable solubilizing agents include wetting agents such as polysorbates, glycerin and poloxamers, non-ionic and ionic surfactants, food acids and bases (e.g. sodium bicarbonate), and alcohols, and buffer salts for pH control. In a preferred embodiment the pH is adjusted using hydrochloric acid (HCL) or sodium hydroxide (NaOH). The pH of the injectable formulation is typically between about 6.9-7.4, preferably about 7.0

Stabilizers are used to inhibit or retard drug decomposition reactions which include, by way of example, oxidative reactions. A number of stabilizers may be used. Suitable stabilizers include polysaccharides, such as cellulose and cellulose derivatives, and simple alcohols, such as glycerol (or glycerin, or glycerine); bacteriostatic agents such as phenol, benzyl alchohol, meta-cresol (m-cresol) and methylparaben; isotonic agents, such as sodium chloride, glycerol (or glycerin, or glycerine), and glucose; lecithins, such as example natural lecithins (e.g. egg yolk lecithin or soya bean lecithin) and synthetic or semisynthetic lecithins (e.g. dimyristoylphosphatidylcholine, dipalmitoylphosphatidylcholine or distearoyl-phosphatidylcholine; phosphatidic acids; phosphatidylethanolamines; phosphatidylserines such as distearoyl-phosphatidylserine, dipalmitoylphosphatidylserine and diarachidoylphospahtidylserine; phosphatidylglycerols; phosphatidylinositols; cardiolipins; sphingomyelins.

In one example, the stabilizer may be a combination of glycerol, bacteriostatic agents and isotonic agents. The most preferred formulations include glycerine and m-cresol. The range for glycerin is about 1-35 mg/ml, preferably about 10-25 mg/ml, most preferably about 19.5-22.5 mg/ml. The range for m-cresol is about 0.75-6 mg/ml, preferably about 1.8-3.2 mg/ml, most preferably about 2 or 3 mg/ml. Calcium chloride can be added to the formulation to “neutralize” any free EDTA and sodium citrate and/or citric acid is added to stabilize the dissociated monomer. Calcium chloride is more typically added to the formulation when the chelator is disodium EDTA. It is added in matched approximately equimolar concentration to the disodium EDTA. For example, if the disodium EDTA is 5 mM, then 5 mM calcium chloride should be used. The effective range is 80-120% of disodium EDTA. Other possible candidates for this are magnesium and zinc, that are added in similar quantities. The range for calcium chloride is about 0.1-10 mM, preferably more preferably about 2.5-7.5 mM, most preferably about 5 mM.

In some embodiments, commercial preparations of insulin and insulin analogs preparations can be used as the insulin of the formulations disclosed herein. Therefore, the final formulation can include additional excipients commonly found in the commercial preparations of insulin and insulin analogs, including, but not limited to, zinc, zinc chloride, phenol, sodium phosphate, zinc oxide, disodium hydrogen phosphate, sodium chloride, tromethamine, and polysorbate 20. These may also be removed from these commercially available preparations prior to adding the chelator and dissociating/stabilizing agents described herein.

Examples of formulations are described in detail in the Examples below. A preferred formulation includes 100 U/ml of insulin, 1.8 mg/ml of calcium disodium EDTA, 2.7 mg/ml of citric acid, 20.08 mg/ml of glycerin, and 3.0 mg/ml of m-cresol (“BIOD-105” of Table 1). Another preferred formulation includes 100 U/ml of insulin or an insulin analog, 1.8 mg/ml of disodium EDTA, 2.7 mg/ml of citric acid, 18.1 mg/ml of glycerin, 2.0 mg/ml of m-cresol, and 5 mM of calcium chloride (“BIOD-107” of Table 1).

### III. Methods of Making the Formulations

In a preferred embodiment, the injectable formulation contains insulin, disodium or calcium disodium EDTA, citric acid, saline or glycerin, m-Cresol and optionally calcium chloride. Typically, calcium chloride is not needed when the EDTA is a calcium disodium EDTA. In the most preferred embodiment, the subcutaneous injectable formulation is produced by combining water, disodium EDTA, citric acid, glycerin, m-Cresol and insulin by sterile filtration into multi-use injection vials or cartridges.

Methods of making the Injectable insulin formulations are described in detail in the Examples below.

In one embodiment, the EDTA is added to the formulation(s) prior to the citric acid. In one embodiment, sodium citrate is added instead of citric acid. In the preferred embodiment, citric acid is added to the formulation(s) prior to the EDTA.

In one preferred embodiment the components of the formulation are added to water: citric acid, EDTA, glycerin, m-Cresol, calcium chloride (optionally) and insulin. Glycerol and m-Cresol are added as a solution while citric acid, EDTA and calcium chloride may be added as powder, crystalline or pre-dissolved in water

In some embodiments, the subcutaneous injectable formulation is produced my mixing water, citric acid, EDTA, glycerin and m-Cresol to form a solution (referred to as the “diluent”) which is filtered and sterilized. The insulin is separately added to water, sterile filtered and a designated amount is added to a number of separate sterile injection bottles which is then lyophilized to form a powder. The lyophilized powder is stored separately from the diluent to retain its stability. Prior to administration, the diluent is added to the insulin injection bottle to dissolve the insulin and create the final reconstituted product.

After the predetermined amount of insulin is subcutaneously injected into the patient, the remaining insulin solution may be stored, preferably with refrigeration.

In another embodiment, the insulin is combined with the diluent, pH 4, sterile filtered into multi-use injection vials or cartridges and frozen prior to use.

After the predetermined amount of insulin is subcutaneously injected into the patient, the remaining insulin solution may be stored, preferably with refrigeration. Alternatively, the insulin solution may be frozen prior to use.

In a preferred embodiment, the insulin is prepared as an aqueous solution at about pH 7.0, in vials or cartridges and kept at 4° C.

### IV. Methods of Modulating Insulin Absorption

The concentration of chelator can be used to optimize the pharmacokinetics and pharmacodynamics of the insulin formulations following subcutaneous injection. As described in Example 1 below, 1.8 mg/ml EDTA in an rapid acting insulin formulation results in an rapid insulin absorption profile (Cmax, Tmax, and ½ Tmax) and pharmacodynamic action (time to decrease plasma glucose 20 mg/dL and time to reach nadir (lowest point)). Concentrations of EDTA lower than 1.8 mg/ml decrease Cmax (maximum concentration of insulin in the plasma) and delay the time to Tmax (time after administration when the maximum concentration is reached) and ½ Tmax, changing the absorption profile of insulin to one that is less peaked. In addition, lower concentrations of EDTA result in a longer response time (time for glucose to drop 20 mg/dL) and longer time to reach nadir.

In other embodiments, calcium disodium EDTA is substituted for disodium EDTA to reduce site reaction. This direct substitution of EDTA modulates the insulin action by delaying the rapid absorption of insulin. To reduce this effect, disodium EDTA and calcium chloride may be used in combination with an increased concentration of citrate ions.

### V. Stability Enhancement

Stability of the formulations can be further optimized by reduction in the m-cresol content and adding additional citrate ions (citric acid) to the formulation.

### VI. Methods of Using Formulations

The formulations may be injected subcutaneously or intramuscularly. The formulation is designed to be rapidly absorbed and transported to the plasma for systemic delivery.

Formulations containing insulin as the active agent may be administered to type 1 or type 2 diabetic patients before or during a meal. Due to the rapid absorption, the compositions can shut off the conversion of glycogen to glucose in the liver, thereby preventing hyperglycemia, the main cause of complications from diabetes and the first symptom of type 2 diabetes. Currently available, standard, subcutaneous injections of human insulin must be administered about one half to one hour prior to eating to provide a less than desired effect, because the insulin is absorbed too slowly to shut off the production of glucose in the liver. A potential benefit to this formulation with enhanced pharmacokinetics may be a decrease in the incidence or severity of obesity that is a frequent complication of insulin treatment.

The present invention will be further understood by reference to the following non-limiting examples.

dissolution/stabilization agent

### EXAMPLE 1

### Comparison of Different EDTA Concentrations in EDTA-Citric Acid Insulin Formulations in Diabetic Swine Study

The purpose of this swine study was to further understand the importance of EDTA in VIAject®. VIAject® was formulated with different concentrations of EDTA and studied in vivo in the diabetic miniature swine model. The reduced EDTA variations were compared to the original formulation containing 1.8 mg disodium EDTA/ml in the diabetic miniature swine model. Results of this testing confirm the importance of EDTA in the formulation.

Materials and Methods

VIAject® U-100 pH 7 formulation (VJ7) includes 100 U/ml insulin, 1.8 mg/ml citric acid, glycerol and m-cresol, and either
(1) 1.8 mg/ml disodium EDTA (VJ7),
(2) 1 mg/mL disodium EDTA (VV1),
(3) 0.25 mg/mL disodium EDTA (VV3)
(4) 0.1 mg/mL disodium EDTA (VV4).

Eight diabetic miniature swine were injected in the morning with 0.25 U/kg of test formulation instead of their daily porcine insulin. Animals were fed 500 g of swine diet and plasma samples were collected at −30, −20, −10, 0, 5, 10, 15, 20, 30, 45, 60, 75, 90, 120, 150, 180, 240, 300 and 360 min post dose using a Becton Dickinson K₂EDTA vacutainer. Frozen plasmas were assayed for insulin content (#EZHI-14K Millipore, USA) and analyzed for glucose concentration (YSI 3200 analyzer, YSI Life sciences, USA).

Basic pharmacokinetic parameters Cmax, Tmax, ½ Tmax and duration were estimated without non-linear modeling. A t-test was performed on the data from each formulation compared to VJ7. Pharmacodynamic response was calculated from the time post dose required to drop the blood glucose level 20 points from baseline and the time to reach nadir.

Results

Mean pharmacokinetic parameters for all eight swine are shown in Table 1. As the EDTA concentration is reduced, the Cmax trends lower while the Tmax increases. There is also a lengthening of the ½ Tmax.

TABLE 1
Mean pharmacokinetic parameters for eight swine given insulin
formulations with reduced concentrations of EDTA.

| +/−SEM | VJ7 | VV1 | VV3 | VV4 |
|---|---|---|---|---|
| Cmax | 124.3 ± 14.3 | 106.7 ± 20.3 | 89.3 ± 14.8 | 91.7 ± 11.3 |
| (μU/ml) | | | | |
| Tmax | 20.6 ± 2.2 | 25.6 ± 5.0 | 34.4 ± 8.3 | 31.2 ± 6.0 |
| (min) | | | | |
| ½ Tmax | 6.9 ± 1.2 | 7.3 ± 1.6 | 9.8 ± 1.7 | 10.7 ± 2.5 |
| (min) | | | | |

EDTA content key:
VJ7 = 1.8 mg/ml,
VV1 = 1.0 mg/ml,
VV3 = 0.25 mg/ml, and
VV 4 = 0.1 mg/ml.

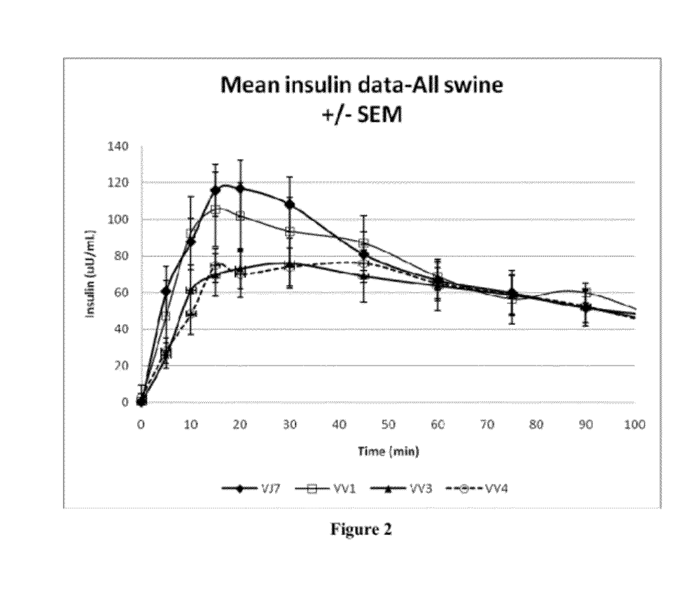

The means of the early concentration versus time profile is shown in FIG. 2. The insulin data show a less peaked profile when less EDTA is in the formulation, although a reduction to 1 mg/mL (VV1) is similar to the original formulation of VJ7.

Pharmacodynamic response as calculated by the time to reduce plasma glucose 20 mg/dL and time to reach nadir is shown in Table 2.

TABLE 2
Pharmacodynamic response: Drop in plasma glucose to 20 mg/dL and
time to reach nadir.

| ±SEM | VJ7 | VV1 | VV3 | VV4 |
|---|---|---|---|---|
| Time to drop | 5.6 ± 0.6 | 10.7 ± 0.7** | 13.0 ± 0.9** | 10 ± 1.3* |
| (min) | | | | |
| Nadir (min) | 56.2 ± 9.7 | 99.4 ± 13.9* | 133.0 ± 40.1 | 163.0 ± 39.2* |

EDTA concentration:
VJ7 = 1.8 mg/ml,
VV1 = 1.0 mg/ml,
VV3 = 0.25 mg/ml, and
VV 4 = 0.1 mg/ml
*p < 0.05,
**p < 0.001 compared to VJ7

As the EDTA concentration is reduced, the time it takes for the glucose to drop 20 points increases to over 10 min, and the nadir progressively takes longer to achieve.

Conclusion:

The reduction in the amount of EDTA results in a lowering of Cmax and a less rapid absorption of insulin, as demonstrated by a delayed Tmax and ½ Tmax. Pharmacodynamically, the time to a 20 point blood glucose drop was increased from 5 to 10 minutes and the estimated time to glucose nadir was progressively retarded as the EDTA concentration was reduced from 1.8 mg/mL to 0.1 mg/mL. The study demonstrated a systematic relationship between the concentration of EDTA and the speed of insulin absorption.

### EXAMPLE 2

### Summary of Effect of Calcium disodium EDTA Concentration on Injection Site Discomfort in Humans

Materials and Methods

Each milliliter of Viaject 7 contains 3.7 mg (100 IU) of recombinant human insulin, 1.8 mg of citric acid, 1.8 mg of disodium EDTA, 22.07 mg of glycerin, 3.0 mg of m-Cresol as a preservative, and sodium hydroxide and/or hydrochloric acid to adjust the pH to approximately 7.

Each milliliter of BIOD 102 contains 3.7 mg (100 IU) of recombinant human insulin, 1.8 mg of citric acid, 2.4 mg of calcium disodium EDTA, 15.0 mg of glycerin, 3.0 mg of m-cresol as a preservative, and sodium hydroxide and/or hydrochloric acid to adjust the pH to approximately 7.1.

Each milliliter of -BIOD 103 contains 3.7 mg (100 IU) of recombinant human insulin, 1.8 mg of citric acid, 0.25 mg of disodium EDTA, 2.0 mg of calcium disodium EDTA, 15.0 mg of glycerin, 3.0 mg of m-cresol as a preservative, and sodium hydroxide and/or hydrochloric acid to adjust the pH to approximately 7.1.

Each solution was injected subcutaneously into a human volunteer and the volunteer was asked to rate the pain associated with the injection.

Results

As shown in Table 3, the samples containing calcium disodium EDTA had slightly lower Cmax and later Tmax than the samples containing only disodium EDTA. However, the calcium disodium EDTA had significantly less injection site pain than the disodium EDTA samples (Table 4).

TABLE 3
Comparison of calcium disodium EDTA with disodium EDTA Pharmacokinetic Data

| | | BIOD | Viaject | BIOD 102 vs VJ7 | BIOD 103 vs VJ7 |
|---|---|---|---|---|---|
| Variable | BIOD 102 | 103 | 7 (VJ) | Ratio/Difference (CI) | Ratio/Difference (CI) |
| AUC₀₋₄₈₀ | 10005.6 | 10139.6 | 9844.8 | 1.02 (0.98, 1.06) | 1.03 (0.99, 1.07) |
| Cmax | 54.0 | 53.4 | 66.1 | 0.82 (0.68, 0.98) | 0.81 (0.68, 0.96) |
| T_{50%}Early | 12.9 | 17.3 | 11.0 | 1.9 (−3.0, 6.8) | 6.4 (1.8, 11.0) |
| Tmax | 73.1 | 63.9 | 34.2 | 38.9 (17.0, 60.8) | 29.7 (9.0, 50.1) |
| T_{50%}Late | 210.6 | 206.4 | 116.4 | 94.2 (49.6, 138.8) | 90.0 (48.2, 131.7) |

TABLE 4
Injection Site Discomfort Data

| | | | | BIOD 102 | BIOD 103 |
|---|---|---|---|---|---|
| | | | Viaject 7 | vs VJ7 | vs VJ7 |
| Variable | BIOD 102 | BIOD 103 | (VJ7) | p-value | p-value |
| VAS | 7.7 | 12.4 | 21.0 | 0.026 | 0.109 |
| Severity | 0.55 | 0.56 | 1.10 | 0.030 | 0.025 |
| Relative | 2.84 | 2.98 | 3.58 | 0.023 | 0.244 |

Viaject 7: 1.8 mg of disodium EDTA
BIOD 102: 2.4 mg of calcium disodium EDTA
BIOD 103: 0.25 mg of disodium EDTA, 2.0 mg of calcium disodium EDTA
VAS: 0 = None, 100 = Worst possible
VR Absolute Discomfort: 0 = None, 1 = Mild, 2 = Moderate, 3 = Severe
VR Relative (to usual injections): 1 = Much less, 2 = Less, 3 = Equal, 4 = Increased, 5 = Much increased

### EXAMPLE 3

### Addition of Blend of Na EDTA and CaCl₂ as a Ssubstitution for Ca EDTA. Comparison of BIOD 105 and BIOD 107 to VJ7-Stability Assessment:

The addition of calcium EDTA to the formulation has been shown to reduce the site reaction to the injection, however, the rapid action of the formulation was somewhat delayed from this substitution. Therefore, new formulations were developed to regain the loss in timing and improve stability. Additional citric acid was added (150% compared to the original formulation, VJ 7) and a ⅓ reduction in m-cresol was also explored to enhance stability. In one new formulation, disodium EDTA and CaCl₂ were added as separate excipients to achieve the calcium chelated form of EDTA (BIOD 107) and this effect was compared to the direct addition of Ca EDTA (BIOD 105). The composition of the formulations given as percents compared to the original formulation, VJ7 are given in Table 5 below.

TABLE 5
Compositions of Na and Ca EDTA formulations:

| | Composition (vs VJ 7) | | | | Additives | |
|---|---|---|---|---|---|---|
| | NaEDTA | CaEDTA | Citric Acid | Glycerin | m-cresol | CaCl2 |
| Formulation No. | (%) | (%) | (%) | (%) | mg/mL | mM |
| VJ 7 | 100 | | 100 | 100 | 3.0 | |
| BIOD-105 | | 100 | 150 | 91 | 3.0 | |
| BIOD-107 | 100 | | 150 | 82 | 2.0 | 5 |

NaEDTA indicates disodium EDTA and
CaEDTA indicates calcium disodium EDTA.

Conclusions:

The most preferred formulations are BIOD-105 and BIOD-107. The slight reduction in m-cresol and addition of CaCl₂ and disodium EDTA extended the shelf life (BIOD 107).

### EXAMPLE 4

### Study of the Rate of Insulin Absorption of Formulations BIOD 105 and BIOD 107 in Miniature Diabetic Swine

Previous clinical studies have shown an association with local injection site discomfort following subcutaneous (sc) administration of recombinant human (RHI), disodium EDTA and citric acid, which has an ultra-rapid onset of action in man when compared to RHI or insulin lispro. The aim of the present study was to evaluate the pharmacokinetic (PK) and pharmacodynamic (PD) properties of several modified formulations predicted to be associated with improved toleration.

Methods and Materials

Six to eight diabetic miniature swine were injected in the morning with 0.25 U/kg of test formulation instead of their daily porcine insulin. Animals were fed 500 g of swine diet and plasma samples were collected at −30, −20, −10, 0, 5, 10, 15, 20, 30, 45, 60, 75, 90, 120, 150, 180, 240, 300 and 360 min post dose using a Becton Dickinson K₂EDTA vacutainer. Frozen plasmas were assayed for insulin content (#EZHI-14K Millipore, USA) and analyzed for glucose concentration (YSI 3200 analyzer, YSI Life sciences, USA). The formulations BIOD 105 and 107 were subcutaneously injected into miniature swine. Comparisons of the rate of absorption were done to determine improvement in the rapid absorption.

Basic pharmacokinetic parameters Cmax, Tmax, ½ Tmax and duration were estimated without non-linear modeling. A t-test was performed on the data from each formulation compare to VJ7. Absorption rate was calculated as the slope of line drawn from the initial increase in insulin concentration post injection (up to 30 min. post dose). Pharmacodynamic response was calculated from the time post dose required to drop the blood glucose level 20 points from baseline and the time to increase 20 points from nadir. The time between these parameters is defined as duration.

Results:

Absorption rate parameters are shown below in Table 6:

TABLE 6
Comparison of the initial rate of absorption of formulations BIOD 105 and
BIOD 107 to the original formulation VJ7.

| | Abs. Rate | Time to drop |
|---|---|---|
| | (uU/mL/min) | (min.) |
| VJ 7 | 5.9 ± 1.6 | 7.4 ± 2.8 |
| BIOD-105 | 4.9 ± 2.2 | 8.5 ± 2.0 |
| BIOD-107 | 4.6 ± 1.5 | 5.5 ± 2.6 |

T-test comparisons show that there is no statistical difference in the initial rate of absorption of these formulations.

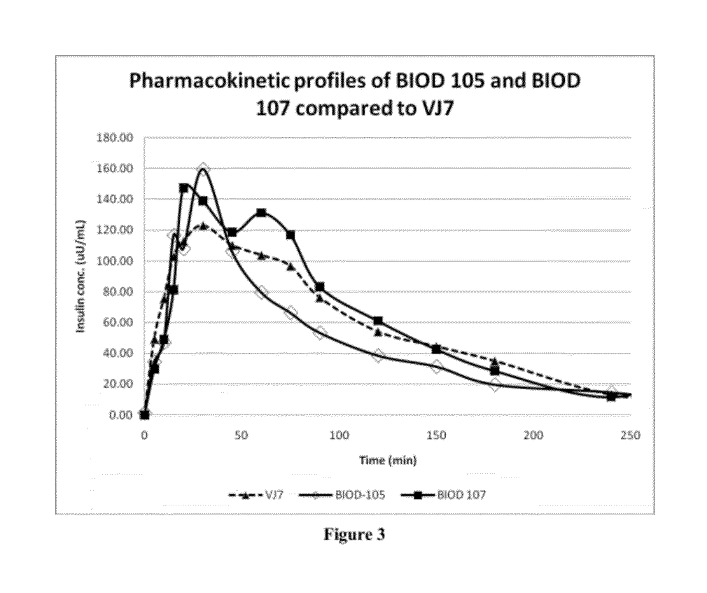

Concentration versus time profiles are seen in FIG. 3. It may be noted that the shape of the curves are slightly different, however, the initial rate of absorption curves are mostly superimposable. This ensures that the onset of insulin action is rapid.

Pharmacodynamic results are shown in Table 7 below.

TABLE 7
Pharmacodynamic parameter calculation.

| | VJ7 | BIOD 105 | BIOD 107 |
|---|---|---|---|
| Time to 20 pt drop | 7.0 ± 1.1 | 8.6 ± 0.8 | 5.5 ± .9 |
| (min.) | | | |
| Time to 20 pt | 193.3 ± 47.0 | 222.4 ± 67.3 | 186.3 ± 39.3 |
| recovery (min.) | | | |
| Duration (min) | 186.8 ± 47.2 | 213.7 ± 67.6 | 180.8 ± 38.7 |

The data shows pharmacokinetically and pharmacodynamically absorption profiles similar to the original formulation are achieved, despite substitution of disodium EDTA with calcium disodium EDTA and increasing in citrate ions.

We claim:

1. An injectable insulin formulation comprising an effective amount of a dissolution/stabilizing agent and an effective amount of calcium or calcium and sodium EDTA to chelate the zinc in the insulin with less injection site discomfort than the formulation with sodium EDTA.

2. The formulation of claim 1 wherein the insulin is human recombinant insulin.

3. The formulation of claim 1, wherein the chelator is disodium ethylenediaminetetraacetic acid and/or calcium disodium ethylenediaminetetraacetic acid in a range of about 5.5×10⁻²M to about 7×10⁻²M.

4. The formulation of claim 1, wherein the chelator is disodium EDTA, the formulation further comprising CaCl₂

5. The formulation of claim 1, wherein the chelator is disodium ethylenediaminetetraacetic acid and/or calcium disodium ethylenediaminetetraacetic acid in a range of about 3.0×10⁻³M to about 1.2×10 ⁻²M.

6. The formulation of claim 1, wherein the chelator is disodium ethylenediaminetetraacetic acid and/or calcium disodium ethylenediaminetetraacetic acid at about 6.0×10⁻²M.

7. The formulation of claim 1 wherein the dissolution/stabilization agent is selected from the group consisting of acetic acid, ascorbic acid, citric acid, glutamic, succinic, aspartic, maleic, fumaric, adipic acid, and salts thereof.

8. The formulation of claim 1 wherein the dissolution/stabilization agent forms citric ions and the pH is about 7.

9. The formulation of claim 7 wherein the dissolution/stabilization agent is citric acid or sodium citrate.

10. The formulation of claim 8 wherein the dissolution/stabilization agent is citric acid or sodium citrate in a range of 2.0×10⁻⁴ M to 4.5×10⁻³M.

11. The formulation of claim 1 wherein the dissolution/stabilization agent is citric acid or sodium citrate in a range of 7×10⁻³M and 2×10⁻² M.

12. The formulation of claim 1 wherein the dissolution/stabilization agent is citric acid or sodium citrate at about 9.37×10⁻³M or about 1.4×10⁻² M.

13. The formulation of claim 1 further comprising calcium chloride.

14. The formulation of claim 1 further comprising glycerine and m-cresol.

15. An insulin formulation comprising 100 U/ml of human recombinant insulin, about 2.7 mg/ml anhydrous citric acid, about 1.8 mg/ml calcium disodium EDTA, about 18 mg/ml of glycerin, and about 3.0 mg/ml of m-cresol at a pH of about 7.0.

16. An insulin formulation comprising 100 U/ml of human recombinant insulin, about 1.8 mg/ml of disodium EDTA, about 2.7 mg/ml of anhydrous citric acid, about 18.1 mg/ml of glycerin, about 2.0 mg/ml of m-cresol, and about 5 mM of calcium chloride at a pH of about 7.0.

17. A method of treating a diabetic individual comprising injecting into the individual an effective amount of the injectable insulin formulation selected from the group consisting (a) 100 U/ml of human recombinant insulin, about 1.8 mg/ml of disodium EDTA, about 2.7 mg/ml of anhydrous citric acid, about 18.1 mg/ml of glycerin, about 2.0 mg/ml of m-cresol, and about 5 mM of calcium chloride at a pH of about 7.0 and (b) 100 U/ml of human recombinant insulin, about 2.7 mg/ml anhydrous citric acid, about 1.8 mg/ml calcium disodium EDTA, about 18 mg/ml of glycerin, and about 3.0 mg/ml of m-cresol at a pH of about 7.0.

18. A method of decreasing injection site pain a diabetic individual comprising injecting the individual with an effective amount of an injectable insulin formulation comprising insulin, an effective amount of a dissolution/stabilization agent, and an effective amount of calcium disodium EDTA or calcium and sodium disodium EDTA in combination with calcium chloride to chelate the zinc in the insulin.
